(19) United States
(12) Patent Application Publication
(10) Pub. No.: US 2026/0060608 A1
(43) Pub. Date: Mar. 5, 2026

(54) Systems and Methods for Template-Assisted Pie-Crusting with Digital Image Correlation

(71) Applicant: Endeavor Health Clinical Operations, Evanston, IL (US)

(72) Inventors: Farid Amirouche, Highland Park, IL (US); Hristo Piponov, Deerfield, IL (US); Jason Koh, Winnetka, IL (US)

(21) Appl. No.: 19/311,595

(22) Filed: Aug. 27, 2025

Related U.S. Application Data
(60) Provisional application No. 63/688,111, filed on Aug. 28, 2024.

(51) Int. Cl. A61B 5/00; A61B 5/11; A61B 34/00; A61B 34/30; G06T 7/00; G16H 50/20

(52) U.S. Cl. CPC A61B 5/4533; A61B 5/0082; A61B 5/1127; A61B 5/7246; A61B 34/30; G06T 7/0014; G16H 50/20; A61B 2034/252; A61B 2505/05; G06T 2200/24; G06T 2207/10024; G06T 2207/30004

(57) ABSTRACT

Systems and methods for determining the fiber strain of a subject's ligament are provided. An example method may include capturing, via an imaging device, image data comprising two or more images of the ligament. The method may include determining, by one or more processors, a fiber strain of at least a portion of the ligament by analyzing the image data using digital image correlation, and based on the fiber strain, providing, by the one or more processors, guidance for performing a guided release of the ligament. The guided release may be performed using a template having a plurality of apertures, via which one or more perforations to the ligament of the subject are made using a perforation device. When applied to total knee replacement (TKA) procedures, the guided release techniques ensure optimal balance in the knee throughout both flexion and extension movements and enhances overall functionality of the knee joint.

### CROSS-REFERENCE TO RELATED APPLICATIONS

This application claims priority to U.S. Provisional Application No. 63/688,111 filed Aug. 28, 2024, the entire contents of which are hereby incorporated by reference.

### FIELD OF THE DISCLOSURE

The present disclosure generally relates to systems and methods for determining strain, and more particularly, to systems and methods for using digital image correlation (DIC) to systems and methods for determining the strain of a ligament and providing guidance when releasing the strain via one or more perforations.

### BACKGROUND

In total knee arthroplasty (often referred to as total knee replacement), soft tissue balancing of the medial collateral ligament (MCL) is frequently necessary to optimize the knee's flexion and extension gaps to provide adequate joint stability and range of motion. Moreover, balanced soft tissue achieves significant clinical gains for the patient more quickly than unbalanced soft tissue, thereby improving the patient's clinical outcome.

Soft tissue balancing proves more challenging in patients with varus and valgus knee deformations, which, unfortunately, are both common types of deformities present in patients undergoing total knee arthroplasty. In varus knee deformity, the medial soft tissues are tightened and the lateral structures are weakened, requiring a release of the medial soft tissue structures to achieve balance. The medial soft tissues are tightened, and the lateral structures are weakened, requiring a release of the medial soft tissue structures to achieve balance. In valgus knee deformity, lateral soft tissue structures are tightened which is associated with attenuation of the MCL, exacerbating the complexity of soft tissue balancing procedures. Moreover, varus and valgus knee deformities make it difficult to quantify and attain reproducible soft tissue balance using traditional soft tissue release methods.

Pie-crusting is a soft tissue release method that perforates the MCL using one or more needle punctures to lengthen the MCL fibers, thereby affecting the MCL strain. While the pie-crusting provides a more gradual soft tissue lengthening as compared to traditional methods that sever MCL fibers to achieve an “all-or-nothing” soft tissue release, the location and quantity of the perforations is left to the discretion of the surgeon based on their experience and interpretation of how the knee “feels” when moving it through a series of motions that may be indicative of the MCL strain. As a result, the pie-crusting technique is highly subjective and does not produce controlled, repeatable results from patient to patient.

The results of the soft tissue balancing procedure are assessed by measuring the stiffness and elongation of the released soft tissue structures. Traditional sensor-based techniques measure stiffness and elongation at a single location of the ligament, which can indicate changes throughout the entire ligament. Unfortunately, such conventional techniques do not indicate local changes in stiffness or elongation for the individual fibers that comprise the ligament. Thus, conventional MCL measurement methods do not account for spatial variations of fiber strain, which can indicate that the elongation in one location of the released MCL is not consistent with the elongation in other locations of the ligament.

Therefore, there is an opportunity and need for improved systems and methods for determining ligament strain associated with soft tissue balancing.

### BRIEF SUMMARY

In one embodiment, the disclosure provides a method for determining strain in a fiber of a ligament in a subject. The method may include capturing, via an imaging device, image data comprising two or more images of the ligament; determining, by one or more processors, a fiber strain of at least a portion of the ligament by analyzing the image data using digital image correlation (DIC); and based on the fiber strain, providing, by the one or more processors, guidance for performing a guided release of the ligament, wherein the guided release is performed using a template having a plurality of apertures via which one or more perforations to the ligament of the subject are performed utilizing a perforation device. The method may include additional, fewer, or alternative functionality or actions, including those discussed elsewhere in this document.

In a variation of the embodiment, the plurality of apertures are equidistantly located within the template.

In another variation of the embodiment, providing the guidance may include obtaining, by the one or more processors, an amount of release to achieve via the one or more perforations; and providing, by the one or more processors, at least one of a quantity of the one or more perforations, or a location of the one or more perforations to achieve the amount of release.

In yet another variation of the embodiment, providing the at least one of the quantity of the one or more perforations, or the location of the one or more perforations may include: providing, by the one or more processors, an indication of apertures of the template to utilize when performing the guided release.

In still yet another variation of the embodiment, obtaining the amount of release may include providing, by the one or more processors, the fiber strain to a surgical robotic system; and receiving, by the one or more processors from the surgical robotic system, the amount of release.

In a variation of the embodiment, obtaining the amount of release may include providing, by the one or more processors, the fiber strain to a user interface presented on a display device; and obtaining, by the one or more processors, the amount of release from the user interface.

In another variation of the embodiment, providing the fiber strain to the user interface may include providing, by the one or more processors to the user interface, a visual representation of the fiber strain of one or more fiber bundles comprising the ligament.

In yet another variation of the embodiment, the visual representation may include color-coding indicative of local strain amounts overlaid onto the one or more fiber bundles.

In still yet another variation of the embodiment, providing the guidance may include analyzing, by the one or more processors, the image data to determine one or more physiological characteristics of the subject; and based on the one or more physiological characteristics, providing, by the one or more processors, a look up table correlating an amount of release to at least one of: a quantity of the one or more perforations, or a location of the one or more perforations to achieve the amount of release.

In a variation of the embodiment, the physiological characteristics may include one or more of the following: a length of the ligament, a thickness of the ligament, or a width of the ligament.

In another variation of the embodiment, the image data may be captured during a predetermined movement pattern of the subject that activates the ligament.

In yet another variation of the embodiment, the imaging device may include two or more cameras; and the DIC produces a two-dimensional strain data vector for tracked fiducials.

In still yet another variation of the embodiment, the image data is first image data, the fiber strain is a first fiber strain, and the method further include capturing, via the imaging device, second image data comprising two or more images of the ligament after the guided release; and analyzing, by the one or more processors, the second image data to determine a second fiber strain of at least a portion of the ligament.

In a variation of the embodiment, the method may include, based on the second fiber strain, determining, by the one or more processors, that the guided release was successful; and providing, by the one or more processors, an indication of the successful guided release to a user interface.

In another variation of the embodiment, the method may include, based on the second fiber strain, determining, by the one or more processors, that the guided release was unsuccessful; and based on the second fiber strain, providing, by the one or more processors, additional guidance for performing an additional guided release of the ligament, wherein the additional guided release is performed using the perforation device to make one or more additional perforations of the ligament in accordance with the additional guidance.

In yet another variation of the embodiment, analyzing the second image data to determine a second fiber strain of at least a portion of the ligament may include comparing, by the one or more processors before the guided release and after the guided release, one or more of: a length of one or more fibers of the ligament, a displacement of the one or more fibers, or a deformation of the one or more fibers.

In another embodiment, the disclosure provides a system for determining fiber strain of ligament of a subject. The system may include an imaging device configured to capture image data comprising two or more images of the ligament; one or more processors; and one or more non-transitory memories storing processor-executable instructions that, when executed by the one or more processors, cause the one or more processors to: determine a fiber strain of at least a portion of the ligament by analyzing the image data using digital image correlation (DIC); and based on the fiber strain, provide guidance for performing a guided release of the ligament, wherein the guided release is performed using a template having a plurality of apertures via which one or more perforations to the ligament of the subject are performed utilizing a perforation device. The system may include additional, less, or alternate functionality, including that discussed elsewhere in this document.

In yet another embodiment, a non-transitory computer-readable medium having processor-executable instructions stored thereon that, when executed by one or more processors, cause the one or more processors to at least: capture, via an imaging device, image data comprising two or more images of a ligament; determine a fiber strain of at least a portion of the ligament by analyzing the image data using digital image correlation (DIC); and based on the fiber strain, provide guidance for performing a guided release of the ligament, wherein the guided release is performed using a template having a plurality of apertures via which one or more perforations to the ligament of the subject are performed utilizing a perforation device.

The instructions may direct additional, less, or alternate functionality, including that discussed elsewhere in this document.

Additional, alternate and/or fewer actions, steps, features and/or functionality may be included in an aspect and/or embodiments, including those described elsewhere herein.

### BRIEF DESCRIPTION OF THE DRAWINGS

The figures described below depict various aspects of the system and methods disclosed therein. It should be understood that each figure depicted represents one embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

Shown in the drawings are arrangements which are presently discussed, it being understood, however, that the present aspects are not limited to the precise arrangements and instrumentalities shown, wherein:

FIG. 1 depicts a block diagram of an exemplary computing environment in which methods and systems for determining fiber strain of a medial collateral ligament are implemented, according to some embodiments.

FIG. 2A depicts a block diagram of an exemplary system for determining fiber strain of a medial collateral ligament, according to some embodiments.

FIG. 2B is a first image depicting an exemplary visual representation of fiber strain of the medial collateral ligament, according to some embodiments.

FIG. 2C is a second image depicting an exemplary template positioned over the medial collateral ligament, according to some embodiments.

FIG. 2D is a third image depicting the medial collateral ligament with exemplary perforations performed using the exemplary template, according to some embodiments.

FIG. 2E is a fourth image depicting an exemplary visual representation of fiber strain of the medial collateral ligament after a guided release, according to some embodiments.

FIG. 2F depicts an exemplary bar graph indicating medial collateral ligament fiber bundle lengths before and after multiple guided releases, according to some embodiments.

FIG. 2G depicts a first exemplary table indicating medial collateral ligament stiffness before and after multiple guided releases, according to some embodiments.

FIG. 2H depicts a second exemplary table indicating a medial femorotibial gap before and after multiple guided releases, according to some embodiments.

FIG. 3 depicts a flow diagram of an exemplary method for determining fiber strain of a medial collateral ligament, according to some embodiments.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative and not as restrictive.

### DETAILED DESCRIPTION

### Overview

The disclosed systems and methods of the present disclosure relate to determining fiber strain of a ligament, such as the medial collateral ligament (MCL). The disclosed techniques may include performing a guided release of the MCL by perforating MCL fibers one or more times using a perforation device (e.g., needle). As generally used herein, the term guided release may also be referred to at times herein as a soft tissue release, soft tissue balancing, pie-crusting, and the like. A template having a plurality of apertures may be overlaid onto the MCL to guide the location of the perforations during the soft tissue release procedure. An imaging device (e.g., a camera) may capture images of the MCL as the subject performs a predefined motion that engages the MCL. A computing device or other processor may be configured to determine a fiber strain of at least a portion of the MCL by analyzing the image data using digital image correlation (DIC). The fiber strain may include absolute fiber strain values, relative fiber strain values (e.g., the change in fiber strain), etc., and other relevant values. Based on the fiber strain, the disclosed techniques may provide guidance for performing a guided release of the MCL.

It should be noted that while the instant disclosure primarily focuses on the guided release of an MCL, the disclosed techniques may be adapted to the guided release of alternative ligaments, fiber bundles, and similar structures. For example, it is envisioned that the instant techniques can be implemented during a guided release of ligaments associated with the elbow, shoulder, hip, ankle, wrist, and other joints.

Unlike conventional pie-crusting techniques in which the surgeon qualitatively assesses a subject and subjectively determines the amount the MCL release and the corresponding number of perforations to achieve that release, the instant techniques that utilize DIC to assess fiber strain provide a quantitative basis for evaluating or determining the amount of ligament release needed and the number of perforations required to achieve the release. Moreover, in conventional approaches, after the surgeon subjectively determined the number of perforations, they also subjectively determined the location of each perforation. On the other hand, the instant techniques involve the use of a template to guide MCL perforations to particular locations, providing controlled and repeatable lengthening and release of MCL fibers. The combination of template-assisted pie-crusting, in conjunction with digital image correlation, improves and advances the field of computer-assisted soft tissue balancing. This approach provides systems, methods, and techniques that yield quantifiable, objective, accurate, and reproducible results in soft tissue balancing, ultimately achieving significantly improved clinical outcomes for the patient.

### Exemplary Computing Environment

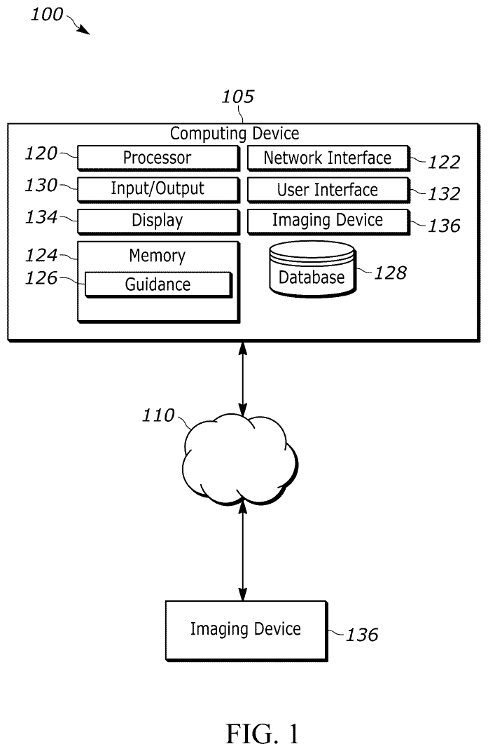

FIG. 1 illustrates an exemplary computing environment 100 used for determining fiber strain of an MCL. Although FIG. 1 illustrates certain entities, components, equipment, and/or devices, it should be appreciated that additional, fewer, and/or alternate entities, components, equipment, and/or devices are envisioned.

The computing environment 100 may include at least one computing device 105 that performs at least some of the functionalities and techniques disclosed for determining MCL fiber strain. The computing device 105 may access services and/or other components of the computing environment 100 via the network 110. The computing device 105 may be and/or include a computer (e.g., desktop computer, laptop computer, terminal, server, a robotic surgical system controller, etc.), a mobile device, augmented reality glasses/headsets, virtual reality glasses/headsets, mixed or extended reality glasses/headsets, and/or other suitable computing device. In at least some embodiments, the computing device 105 may be and/or include a server. The server may be part of a cloud network or may otherwise communicate with other hardware or software components within one or more cloud computing environments to send, retrieve, or otherwise analyze data or information described herein. In some embodiments, the computing environment 100 may comprise an on-premises computing environment, a multi-cloud computing environment, a public cloud computing environment, a private cloud computing environment, and/or a hybrid cloud computing environment. In one example, an entity (e.g., a pharmacy) may host one or more services (e.g., prescription alignment) in a public cloud computing environment (e.g., Amazon Web Services (AWS), Google Cloud, IBM Cloud, Microsoft Azure, etc.). The public cloud computing environment may be a traditional off-premises cloud (i.e., not physically hosted at a location owned/controlled by the business).

Alternatively, or in addition, aspects of the public cloud may be hosted on-premises at a location owned/controlled by the entity. The public cloud may be partitioned using visualization and multi-tenancy techniques and/or may include one or more of the following services: Software-as-a-Service (SaaS), Infrastructure-as-a-Service (IaaS) and/or Platform-as-a-Service (PaaS). In one aspect, the computing device 105 may include a client-server platform technology, such as ASP. NET, Java J2EE, Ruby on Rails, Node.js, a web service or online API, which is responsible for receiving and responding to electronic requests.

The computing device 105 may include at least one processor 120. The processor 120 may include one or more suitable processors (e.g., central processing units (CPUs) and/or graphics processing units (GPUs)). The processor 120 may be communicatively coupled to a memory 124 via a computer bus (not depicted) that transmits electronic data, data packets, or otherwise electronic signals to and from the processor 120 and the memory 124 to execute, implement or perform the machine-readable instructions, methods, processes, elements, or limitations, as illustrated, depicted, or described for the various flowcharts, illustrations, diagrams, figures, and/or other disclosure herein. The processor 120 may interface with the memory 124 to execute an operating system, computing instructions contained therein, and/or to access different services/aspects. For example, the processor 120 may interface with the memory 124 via the computer bus to create, read, update, delete, or otherwise access and interact with the data stored in the memory 124, database 128, and/or another data source.

The computing device 105 may include a network interface 122. The network interface 122 may allow the computing device 105 to communicate over the network 110 via any suitable wired and/or wireless connection, e.g., using any suitable network interface controller(s) of the network interface 122. The network interface 122 may include one or more transceivers (e.g., WWAN, WLAN, and/or WPAN transceivers) functioning by IEEE reference standards, 3GPP reference standards, and/or other reference standards that may be used in receipt and transmission of data via external/network ports of the computing device 105 connected to the computer network 110.

The memory 124 may include one or more forms of volatile, nonvolatile, non-transitory, fixed and/or removable memory, such as read-only memory (ROM), electronic programmable read-only memory (EPROM), random access memory (RAM), erasable electronic programmable read-only memory (EEPROM), hard drives (e.g., hard disk, hybrid, solid state), flash memory, memory cards (e.g., MicroSD), and/or others. The memory 124 may store the operating system (e.g., Microsoft Windows, Linux, UNIX, etc.) capable of facilitating the functionalities, apps, methods, or other software as described herein. The memory 124 may store one or more sets of non-transitory, computer-executable and/or processor-executable instructions that, when executed, cause the computing device 105 to perform certain functions.

In general, a computer program or computer-based product, application, or code may be stored on a computer usable storage medium, or tangible, non-transitory computer-readable medium (e.g., reference random access memory (RAM), an optical disc, a universal serial bus (USB) drive, a hard drive or the like) having such computer-readable program code or computer instructions embodied therein. The computer-readable program code or computer instructions may be installed on, or otherwise adapted to be, executed by the processor 120 (e.g., working in connection with the respective operating system in the memory 124) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language. The program code may be implemented as machine code, assembly code, byte code, interpretable source code or a similar language (e.g., via Golang, Python, C, C++, C#, Objective C, Java, Scala, ActionScript, JavaScript, HTML, CSS, XML, etc.).

The memory 124 may store a Surgical Guidance application 126 that, when executed by the processor 120, performs one or more functions associated with a total knee arthroplasty and/or other soft tissue release procedure, such as causing an imaging device to capture image data of the MCL, determining MCL fiber strain based on image analysis, providing surgical guidance, and/or other suitable functions.

The computing device 105 may include, and/or be communicatively coupled to (e.g., via the network 110), at least one electronic database 128. The database 128 may include a relational database, such as Oracle, DB2, MySQL, a NoSQL database such as MongoDB, and/or another suitable database. The database 128 may store one or more types of data and/or datasets, such as image data. A dataset may include one or more types of data, records, files, etc., and the terms “data”and “dataset”may be used interchangeably herein.

The computing device 105 may include an input/output (I/O) module 130 comprising a set of computer-executable instructions implementing communication functions. The I/O module 130 may include a communication component configured to communicate (e.g., send and receive) data via one or more external/network ports to one or more networks or local terminals, such as the network 110 described herein. The I/O module 130 may include or implement a user interface configured to present information to an administrator, operator, or other user, and/or receive inputs from the user, such as via a touchscreen display. The I/O module 130 may facilitate I/O components (e.g., ports, capacitive or resistive touch-sensitive input panels, keys, buttons, lights, LEDs), which may be directly accessible via, or attached to, the computing device 105 and/or may be indirectly accessible via, or attached to, another device.

The computing device 105 may include at least one user interface 132, which may comprise any type of user interface which is locally provided by the computing device 105. The user interface 132 may include, for example, displays, touchscreens, keyboards, touch pads, mice, microphones, speakers, scanners, cameras, and/or other optical, auditory, and/or physical user interface devices. According to some aspects, a user may access the computing device 105 via the user interface 132 to input data/information, execute the Surgical Guidance application 126, and/or perform other functions. In at least some embodiments, the Surgical Guidance application 126 may provide guidance associated with a surgical procedure via one or more user interfaces 132, such as audio guidance via a speaker, visual guidance via images, application programming interfaces (APIs), etc.

The computing device 105 may include a display 134, such as an LCD, LED, OLED, head-mounted display, etc. A user may view data/information (e.g., MCL fiber strain, surgical guidance) via the display 134. In at least some embodiments, the display 134 may be and/or include a surgical display providing information (e.g., MCL fiber strain) and/or guidance (e.g., number of MCL perforations and their respective locations, correlations between several perforations and an amount of release, etc.) associated with one or more MCL guided releases.

The computing device may include and/or be communicatively coupled to (e.g., via the network 110, the I/O module 130) at least one imaging device 136. The imaging device 136 may be and/or include one or more imaging sensors (e.g., CCD, CMOS), cameras (e.g., digital cameras, stereoscopic cameras), and the like to detect electromagnetic radiation in the visual range or other wavelengths. The imaging device 136 may capture image data comprising one or more still and/or video images of the field of view of the imaging device 136. In at least some embodiments, two or more imaging devices 136 may be disposed to obtain stereoscopic images of the physical environment.

The network 110 may include one or more networks, such as a local area network (LAN), a wide area network (WAN), the Internet, a combination thereof, and/or any other suitable network. Generally, the network 110 enables bidirectional communication between the computing device 105 and other components and/or devices (e.g., other computing devices 105, databases 128, imaging device 136, etc.) within the computing environment 100. In some embodiments, the network 110 may comprise a cellular base station, such as cell tower(s), communicating to the one or more components of the computing environment 100 via wired/wireless communications based on any one or more of various mobile phone standards, including NMT, GSM, CDMA, UMTS, LTE, 5G, 6G, or the like. Additionally, or alternatively, the network 110 may comprise one or more routers, wireless switches, or other such wireless connection points communicating to the components of the computing environment 100 via wireless communications based on any one or more of various wireless standards, including, by non-limiting example, IEEE 802.11 a/ac/ax/b/c/g/n (Wi-Fi), Bluetooth, and/or the like.

In some embodiments, the computing environment 100 may determine fiber strain of the MCL, for example as part of a total knee arthroplasty procedure. A user of the computing device 105 (e.g., a surgeon) may execute the Surgical Guidance application 126, for example, via the user interface 132. The Surgical Guidance application 126, via the computing device 105, may cause one or more imaging devices 136 to capture two or more images, respectively.

In one example, the Surgical Guidance application 126 generates a graphical user interface (GUI) that is displayed on the display 134 of the computing device 105. The GUI may provide the user with various functionalities, including a function for capturing images via the imaging device 136. Upon selection of the image capture function by the user, the Surgical Guidance application 126 may cause the computing device 105 to generate and transmit (e.g., via the network 110, a bus, etc.) a signal (e.g., a command) to the imaging device 136 causing the imaging device 136 to capture two or more images (for example, as a video stream of image data).

In at least some embodiments, the imaging device 136 captures two or more images of at least a portion of the MCL. For example, the subject may be asked to perform a predetermined and/or predefined movement that activates the MCL such that the ligament tenses and/or relaxes. The imaging device 136 may capture image data of the MCL during this process to assess the amount of strain exhibited upon the MCL during the predetermined movement. Accordingly, the imaging device 136 may capture images of the MCL before the guided release to provide guidance related to performance of the guided release and/or after the guided release to determine whether the desired amount of release was achieved. At least a portion of the captured images may be stored in memory (e.g., the memory 124, the database 128, a memory of the imaging device 136, etc.).

The Surgical Guidance application 126 may analyze one or more images of the MCL captured before the guided release (e.g., a first guided release, a second guided release, etc.), and one or more images of the MCL captured after the guided release, to determine the fiber strain of one or more fibers of the MCL. The Surgical Guidance application 126 may determine the MCL fiber strain before the guided release and/or other suitable metrics associated with one or more MCL fibers.

The fiber strain may include one or more values corresponding to different portions of the MCL. For example, a pattern may be applied to the MCL fibers that acts as a fiducial (e.g., a speckle pattern) for tracking through the application of digital image correlation (DIC) techniques. Accordingly, each fiducial may be associated with a respective fiber strain value. That is, during the performance of the predetermined movement, the Surgical Guidance application 126 may track one or more values associated with a length, a displacement, and/or a deformation of one or more MCL fibers as determined based on the fiducials. In some embodiments, an interpolation technique may be applied to calculate fiber strain for portions of the MCL between any set of fiducials. Additionally, in some embodiments, the fiber strain values are determined at multiple times during the performance of the predetermined movement and combined into a composite value for each portion (e.g., by averaging, performing a maximum function, etc.),

It should be appreciated that if the imaging device 136 includes a single image sensor, the fiber strain value may be measured along a single dimension (e.g., along the length of a particular fiber). On the other hand, if the imaging device 136 includes multiple image sensors or is stereoscopic, capturing images of the MCL from different points of view, the strain values may be two-dimensional vectors within a plane defined by the particular fiber. As a result, the Surgical Guidance application 126 may be able to determine additional and/or more accurate MCL strain information for the fiber bundles.

The Surgical Guidance application 126 may cause the computing device 105 to output an indication of the fiber strain of at least the portion of the MCL, for example in real-time during the total knee arthroplasty and/or guided release. The MCL fiber strain may include one or more values and/or metrics, for example, strain values for each of the fiber bundles comprising the MCL. In at least some embodiments, the Surgical Guidance application 126 may generate one or more visual representations of the fiber strain output to a display device (e.g., the user interface 132, the display 134), for example by overlaying color-coding associated with various levels of strain (e.g., local strain amounts) onto a visual representation of one or more fiber bundles of the MCL. In other examples, the visual representation of the fiber strain may be provided in real-time via an augmented reality display, with fiber strain information (e.g., color-coding, strain values, etc.) overlaid onto respective fibers of the MCL. However, the Surgical Guidance application 126 may output any suitable indication of the fiber strain (e.g., a report, a graph, a table, a video, etc.). The Surgical Guidance application 126 may provide additional information associated with the MCL strain, such as MCL fiber lengths, stiffness, and gaps (e.g., medial and/or lateral gaps of the knee in flexion, mid-flexion and extension).

In at least some embodiments, the Surgical Guidance application 126 may generate guidance for the surgeon performing the total knee arthroplasty and/or guided release of the MCL based on the fiber strain. The guidance may include guidance for conducting the guided release (e.g., the number/quantity of perforations, the location of a perforation, the template aperture(s) to utilize when performing the guided release), indicate whether a guided release was successful and/or achieved its intended purpose, whether one or more additional guided releases are suggested, the quantity of perforations to create in the MCL, one or more locations of the MCL perforations, the depth of a perforation, and/or other suitable guidance.

In at least some embodiments, providing guidance may include obtaining (e.g., via the Surgical Guidance application 126, a surgical plan stored in the memory 124, a surgical robotic controller, a user interface, etc.) an amount of release to achieve via one or more perforations, and providing (e.g., via the Surgical Guidance application 126, the display 134, the surgical robotic controller, the user interface, etc.) a quantity of perforations and/or one or more locations of perforations to achieve the amount of release.

Obtaining the amount of release may include providing the fiber strain to a surgical robotic system and obtaining the amount of release from the surgical robotic system; providing the fiber strain via a user interface of a display device; and receiving the amount of release via the user interface. In one example, the Surgical Guidance application 126 may determine that the patient's MCL fiber strain is 24 Newtons per millimeter (N/mm). To achieve a fiber strain 23 N/mm, two perforations may be recommended to the surgeon. The guidance may further indicate that the perforations should be made via the template at the leftmost aperture in the second and third rows of the template.

Providing the guidance may include the Surgical Guidance application 126 analyzing the image data to determine one or more physiological characteristics of the subject (e.g., ligament length, thickness, width) and based on the physiological characteristics, providing a look up table correlating an amount of release to a quantity of perforations and/or a location of the perforation(s) to achieve the amount of release (e.g., absolute positions on the patient physiology and/or particular apertures of the template device that will guide the perforations).

Based on the fiber strain, the Surgical Guidance application 126 may determine that the guided release was either successful or unsuccessful. If successful, the Surgical Guidance application 126 may indicate the successful guided release via a user interface. If unsuccessful, the Surgical Guidance application 126 may provide additional guidance for performing an additional guided release of the ligament.

It should be understood that, while the computing environment 100 is depicted in FIG. 1 as including one computing device 105, one network 110, and two imaging devices 136, different numbers of computing devices 105, networks 110, and/or imaging devices 136 may be utilized.

### Exemplary Determination of MCL Fiber Strain

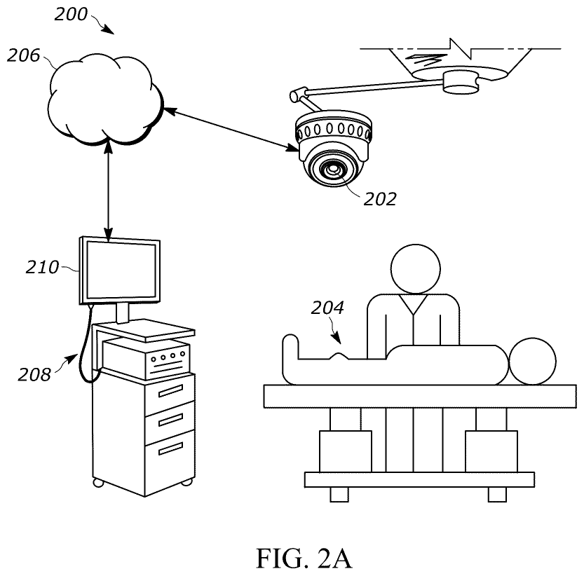

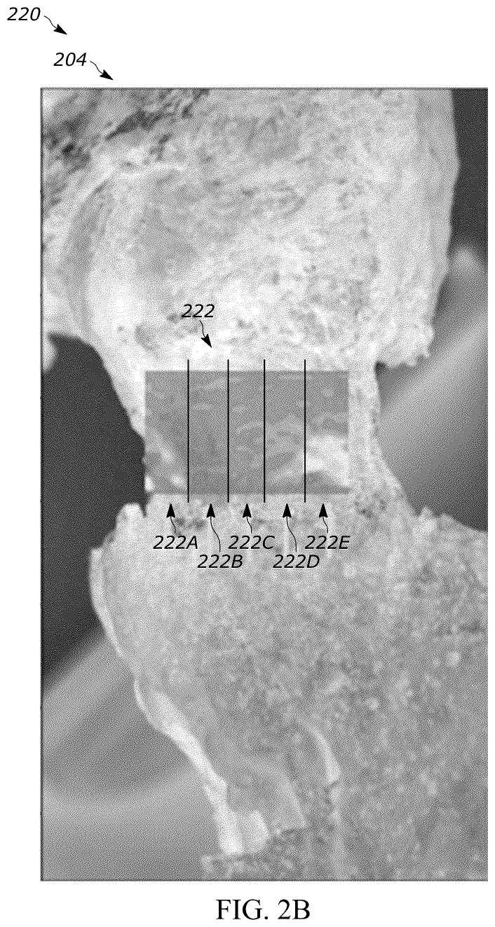

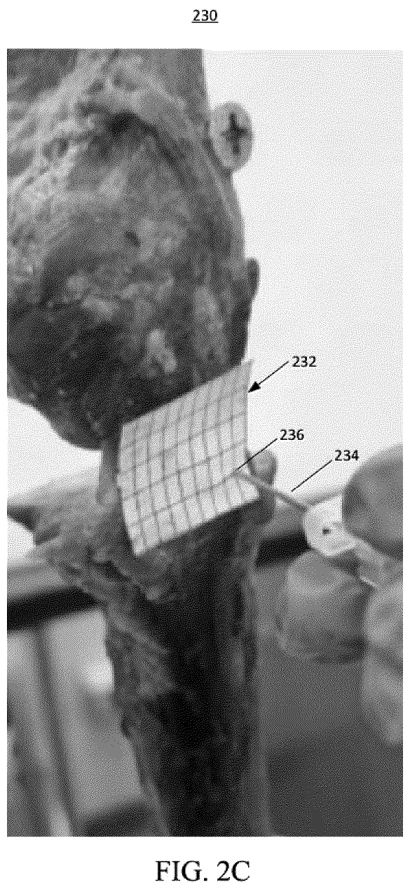

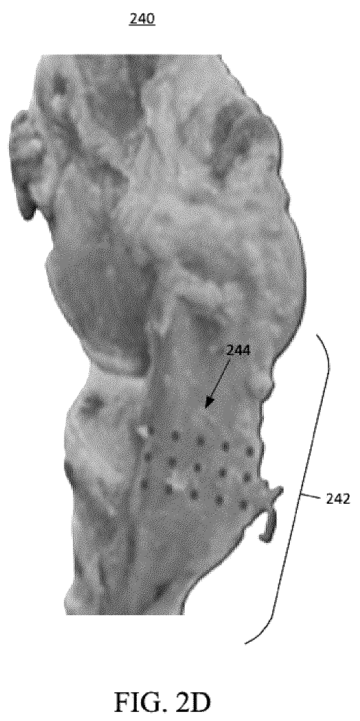

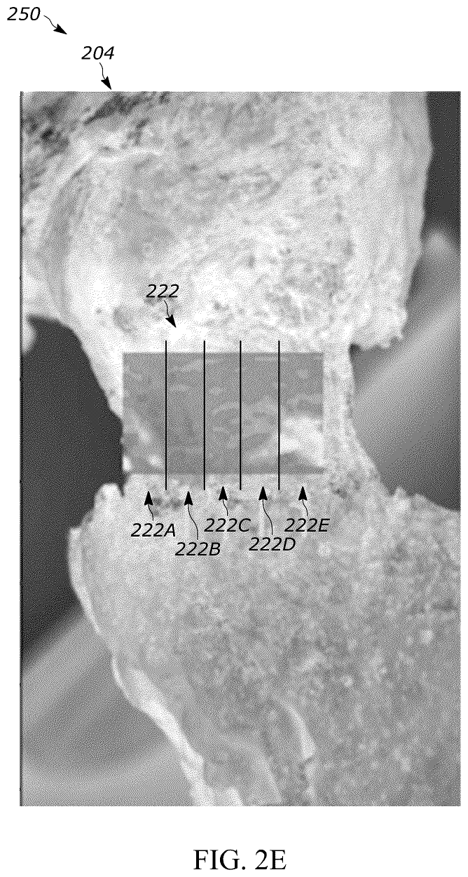

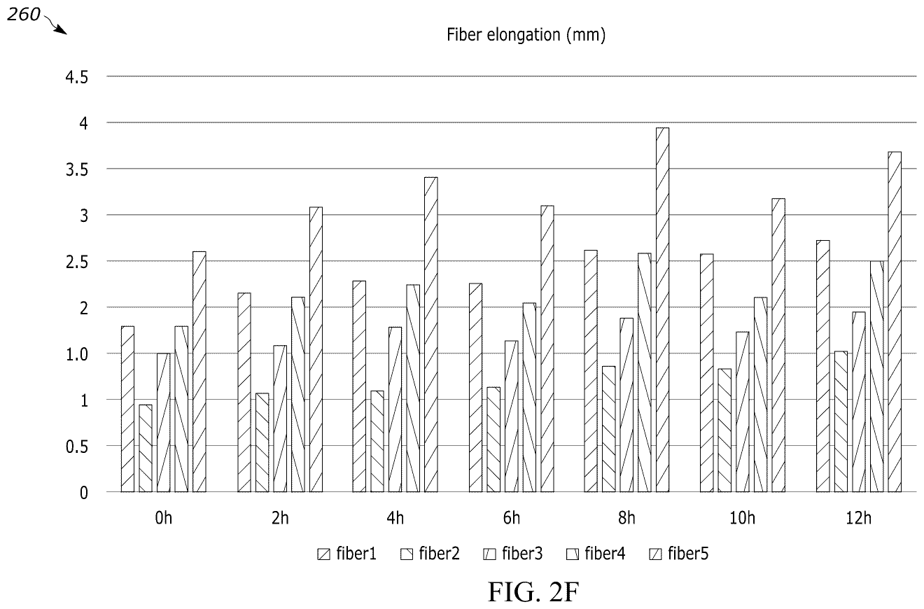

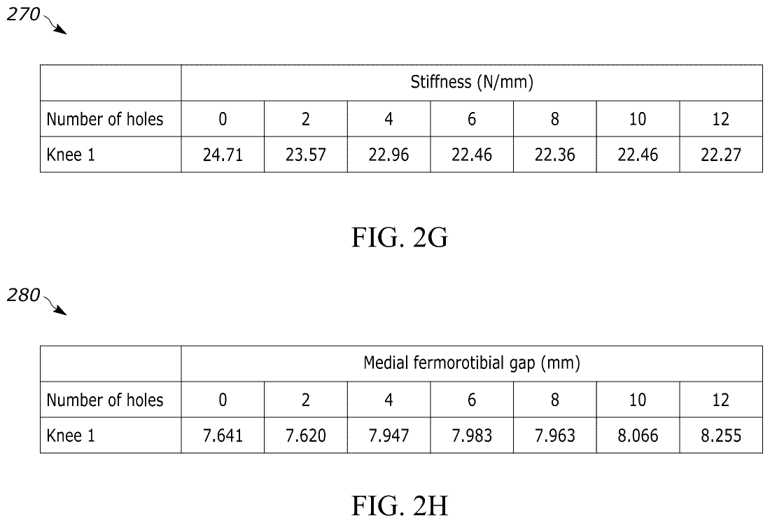

FIG. 2A depicts a block diagram of an exemplary system 200 for determining fiber strain of an MCL, according to some embodiments. The system 200 includes a camera 202 (e.g., the imaging device 136) that is communicatively coupled via a network 206 (e.g., the network 110) to a surgical computer 208 (e.g., the computing device 105), which has a display 210 (e.g., the display 134). The camera 202 is positioned above a surgical bed with a field of view of the MCL of the knee 204 of a patient undergoing a total knee arthroplasty.

During the total knee arthroplasty, the surgeon may perform one or more guided releases of the MCL using a template-assisted pie-crusting method. A random speckle pattern may be applied to the surface of the MCL, where the speckles act as fiducials that are tracked via DIC techniques. The surgical computer 208 may be configured (e.g., via the Surgical Guidance application 126 installed on the surgical computer 208) to detect the fiducials in one or images, for example, using image analysis techniques such as computer vision, object recognition, and machine learning.

The camera 202 may capture first image data comprising one or more images of the MCL before performing any guided release of the MCL. For example, the surgeon may move the knee 204 through a series of positions that cause associated movements of the speckle-coated MCL fibers. The MCL movements may cause changes in the length, displacement, and/or deformation of the MCL fibers. The camera 202 may capture one or more images of the speckle-coated MCL throughout the movements to generate the first image data.

The camera 202 may transmit the first image data to the surgical computer 208 via the network 206. The surgical computer 208, via the Surgical Guidance application 126, may analyze the images to determine the fiber strain of one or more fibers in the MCL. For example, the Surgical Guidance application 126 may determine the MCL fiber strain based on changes in the speckle pattern (e.g., changes in the location, orientation, reflectivity, etc., of the speckles) during the predetermined MCL movements which indicate elongation, displacement, deformation, etc., of the MCL fibers. The Surgical Guidance application 126 may generate one or more indications of the fiber strain of one or more of the MCL fibers. The surgical computer 208 may store the MCL fiber strain indications in memory (e.g., the memory 124, the database 128, etc.) and/or output the MCL fiber strain indication to the display 210.

FIG. 2B is a first image 220 that depicts an exemplary visual representation of the indications of MCL fiber strain in the MCL of the patient's knee 204, according to some embodiments. The Surgical Guidance application 126 may generate the first image 220, and cause the surgical computer 208 to display the first image 220 on the display 210. The Surgical Guidance application 126 may superimpose a shaded overlay 222 on the MCL in the first image 220. The shaded overlay 222 may include five segments 222A-222E, respectively, from left to right. The shaded overlay 222 may indicate the relative strain on the fibers of the MCL using various levels of shading, with darker shading of the MCL fiber indicating less strain than lighter shading.

Based on the strain of the MCL fibers, the Surgical Guidance application 126 may provide guidance suggesting the performance of one or more guided releases of the MCL. The guided release may include applying a template to the MCL. The template may guide the location of perforations caused by a perforation device, such as a needle (e.g., an 18-gauge needle), during pie-crusting of the MCL. The template may indicate locations to perforate the MCL. In one example, the template includes intersecting features, with the intersections indicating MCL perforation locations. In another example, the template may include multiple apertures located along a first direction and/or a second direction that is perpendicular to the first direction, the apertures indicating MCL perforation locations.

In some embodiments, additive manufacturing techniques (e.g., using a three-dimensional printer) may be employed to produce the template. In at least some embodiments, the template may be patient-specific based on the physiological characteristics of the MCL of the patient (e.g., MCL fiber thickness, length, etc.). For example, the template may cover the entire surface of the MCL, with the size of the template being determined based on measurements of the medial femorotibial gap and the width of the MCL at whole knee extension. Accordingly, to produce the template, an image of the relevant patient physiology may be analyzed to derive the appropriate shape of the template when it is stretched to its ultimate shape. In other embodiments, a healthcare facility may have multiple pre-made templates.

The templates may have different sizes and/or include apertures of various sizes for use with varying gauges of perforation devices. Accordingly, the Surgical Guidance application 126 may provide guidance for selecting (or printing) an appropriate template, as well as guidance for selecting (or printing) appropriately sized apertures based on the gauge of the perforation device to be used during the guided release. For example, the Surgical Guidance application 126 may analyze a capture image of the relevant patient physiology to determine which size template best aligns with the patient's ligament and/or the appropriate gauge to use when performing the perforations.

In some embodiments, the produced template is pliable and/or deformable, allowing apertures may be adjusted to a desired shape that better aligns with the patient's physiology. Accordingly, while the template may include a “grid” that forms the apertures, the “grid” can have any suitable shape. For example, the template may be stretchable along one or more axes while retaining the apertures that guide the perforations. In these embodiments, the template may include markings that are to be aligned with corresponding markings on the patient, providing the user with guidance as to the final shape of the template.

The holes in the template may have a diameter of 2.5 mm to ensure a smooth passage for an 18-gauge needle. The apertures of the template may start at the posterior of the MCL at the tibial plateau level and continue in an inferior-to-superior and right-to-left direction, extending up to the transverse femoral component. The MCL apertures may be equidistantly spaced across the template (e.g., apertures spaced every 3 millimeters in both the vertical and horizontal directions). Although the foregoing results in apertures that generally form a grid pattern, the apertures may be arranged in any suitable manner that aligns with the ligament.

FIG. 2C is a second image 230 depicting an exemplary template 232 positioned over the MCL of the knee 204. The template 232 may guide a needle 234 (e.g., perforation device) during the pie-crusting procedure, according to some embodiments. The template 232 includes a first set of lines oriented vertically, and a second set of lines oriented horizontally, intersecting the first set of lines. The locations of the intersections of the first and second sets of lines may be used to guide the needle 234 when creating one or more perforations in the MCL, such as the intersection 236 used to guide the location of the needle 234 while perforating the MCL.

FIG. 2D is a third image 240 depicting the MCL 242 with a set of exemplary perforations 244 performed using the exemplary template 232 during the pie-crusting procedure, according to some embodiments. The set of perforations 244 includes fifteen substantially equidistant perforations including five columns of perforations, with each column having three perforations. Each of the five columns of perforations may coincide, for example, with each of the five segments 222A-222E of the shaded overlay 222.

The camera 202 may capture image data comprising one or more images of the MCL 242 (e.g., throughout various movements) after performing the predefined movement of the MCL, and transmit the image data to the surgical computer 208 (e.g., via the network 206). The Surgical Guidance application 126 may analyze the image data to determine the MCL fiber strain of one or more MCL fibers during the predefined movement. The Surgical Guidance application 126 may determine the absolute MCL fiber strain of one or more MCL fibers, the relative change in MCL fiber strain during the predefined movement, and/or other suitable metrics as further described below.

FIG. 2E is an image 250 depicting an exemplary visual representation of fiber strain of the MCL of the knee 204 after the guided release, according to some embodiments. The Surgical Guidance application 126 may generate the image 250, and/or cause the surgical computer 208 to display the image 250 on the display 210. The shaded overlay 222 indicates the strain of various fibers of the MCL after the guided release.

Fiber strain has a nonuniform distribution across the MCL and is concentrated in specific areas of the ligament with no apparent pattern. As additional perforations 244 are created in the MCL, the local fiber strain changes, as demonstrated by the shaded overlay 222. For example, the rightmost overlay section 222E of the fourth image 250 includes lighter shading as compared to the shading of the overlay section 222E in the first image 220.

The Surgical Guidance application 126 may provide guidance (e.g., via the display 210 and/or other user interface(s) 132 of the surgical computer 208), for example guidance as to whether the guided release was able to achieve the desired amount of release. In one example, the guidance may indicate the locations to perform additional MCL perforations using the template 232. In another example, the guidance may suggest that no additional guided releases are required and/or that the pie-crusting operations already performed are successful.

In at least some embodiments where numerous guided releases are performed, the Surgical Guidance application 126 may determine the MCL strain after one or more of the multiple guided releases using the inventive techniques. The camera 202 may capture one or more sets of additional image data, each additional set comprising one or more images of a perforated MCL after a respective additional guided release. The Surgical Guidance application 126 may determine the (change in) MCL fiber strain of at least the portion of the MCL based on analyzing two or more of the first image data, the second image data, or at least one set of additional image data. The Surgical Guidance application 126 via the surgical computer 208 may output any further indications of MCL fiber strain to the display 210.

The Surgical Guidance application 126 may generate and/or output (e.g., via the surgical computer 208 to the display 210) additional and/or alternate information associated with the MCL, the MCL fiber strain, and the like, such as alternate indications of MCL fiber strain (e.g., numeric values), MCL fiber stress, MCL fiber lengths, MCL fiber stiffness, MCL fiber gaps, etc. For example, the Surgical Guidance application 126 may provide information on the medial and/or lateral gaps of the knee in flexion, mid-flexion and extension, and provide associated guidance for soft tissue balancing during a total knee arthroplasty.

FIG. 2F depicts an exemplary bar graph 260 indicating MCL fiber bundle lengths before and after multiple guided releases, according to some embodiments. The bar graph 260 indicates MCL fiber bundle lengths for each of the five fiber bundles associated with each of the five shaded overlay sections 222A-222E, both before and after six guided releases. The bar graph 260 indicates the number of perforations of each MCL fiber bundle for respective guided releases on the x-axis, and values for the MCL fiber bundle lengths in millimeters on the y-axis. The first set of MCL fiber bundle lengths represents the fiber bundle lengths before the first guided release (i.e., with zero perforations). The second, third, fourth, fifth and sixth sets of fiber bundle lengths indicate the fiber bundle length after a first guided release performing two perforations, a second guided release performing four perforations, a third guided release performing six perforations, a fourth guided release performing eight perforations, a fifth guided release performing ten, and a sixth guided release performing twelve perforations respectively. It should be appreciated that while the bar graph 260 compares the length of fiber bundles, in other embodiments, the elongation for individual fibers of the fiber bundles is also measured.

As previously described, guidance for MCL balancing may be provided as a lookup table indicating a correlation between several perforations and a desired amount of release (e.g., as measured in stiffness or distance). FIG. 2G depicts a first exemplary lookup table 270 indicating a correlation for several holes to an MCL stiffness in Newton/millimeters (N/mm) to be released, according to some embodiments. Similarly, FIG. 2H depicts a second exemplary lookup table 280 indicating a correlation between the number of holes and an increase in the medial femorotibial gap. It should be appreciated that while the lookup tables 270 and 280 compare stiffness/medial femorotibial gap to a number of holes, in other embodiments, the lookup tables 270 and 280 dynamically provide an amount of stiffness and/or medial femorotibial gap based upon a particular arrangement of perforations to be performed using the template device. For example, the Surgical Guidance application 126 may provide a user interface that presents a suggested arrangement of perforations to the user, along with the corresponding values for the stiffness and/or the medial femorotibial gap when that arrangement is applied. In these embodiments, the user may interact with the user interface to adjust the suggested arrangement of perforations, causing the Surgical Guidance application 126 to dynamically recalculate the stiffness and/or medial femorotibial gap based on the user-modified arrangement.

### Exemplary Method for Determining Fiber Strain of an MCL

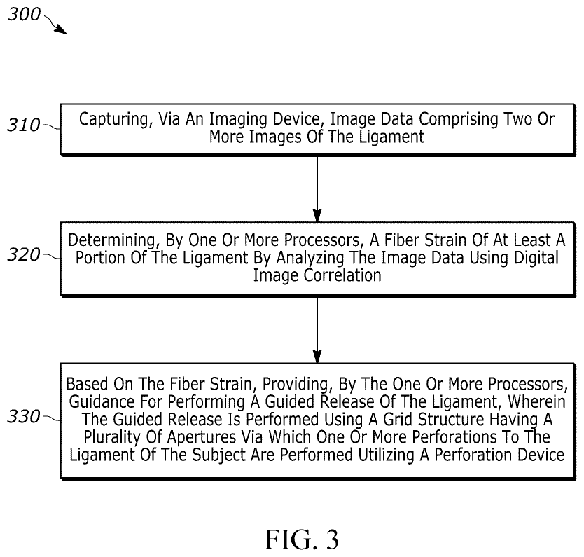

FIG. 3 is a flow diagram depicting an exemplary method 300 for determining fiber strain of a ligament in a patient, according to some embodiments. In general, at least a portion of the method 300 may be performed by the devices, models, and/or other components of the computing environment 100. One or more steps of the method 300 may be implemented as a set of instructions stored on a non-transitory computer-readable memory (e.g., the memory 124) and executable by one or more processors (e.g., the processor 120).

The method 300 may include capturing, via an imaging device (e.g., the imaging device 136), image data comprising two or more images of the ligament (block 310). The imaging device may include two or more cameras (e.g., stereoscopic cameras).

The method 300 may include determining, by one or more processors, a fiber strain of at least a portion of the ligament by analyzing the image data using digital image correlation (DIC) (block 320).

The method 300 may include, based on the fiber strain, providing, by the one or more processors, guidance for performing a guided release of the ligament, wherein the guided release is performed using a template (e.g., the template 232) having a plurality of apertures via which one or more perforations to the ligament of the subject are performed utilizing a perforation device (e.g., the needle 234) (block 330). The plurality of apertures may be equidistantly located within the template.

In at least some embodiments of the method 300, providing the guidance may include obtaining, by the one or more processors, an amount of release to achieve via the one or more perforations; and providing, via the one or more processors, at least one of a quantity of the one or more perforations, or a location of the one or more perforations to achieve the amount of release. In some such embodiments, providing the at least one of the quantity of the one or more perforations, or the location of the one or more perforations may include providing an indication of apertures of the template to utilize when performing the guided release. In some such embodiments, obtaining the amount of release may include providing the fiber strain to a surgical robotic system; and obtaining the amount of release from the surgical robotic system. In some such embodiments, obtaining the amount of release may include providing the fiber strain to a user interface presented on a display device; and obtaining the amount of release from the user interface. Providing the fiber strain to the user interface may include providing a visual representation of the fiber strain of one or more fiber bundles comprising the ligament to the user interface. The visual representation may include color-coding indicative of local strain amounts overlaid onto the one or more fiber bundles.

In at least some embodiments of the method 300, providing the guidance may include analyzing, by the one or more processors, the image data to determine one or more physiological characteristics of the subject; and based on the one or more physiological characteristics, providing, by the one or more processors, a look up table correlating an amount of release to at least one of the following: a quantity of the one or more perforations, or a location of the one or more perforations to achieve the amount of release. The physiological characteristics may include one or more of a length of the ligament, a thickness of the ligament, or a width of the ligament.

In at least some embodiments of the method 300, the image data may be captured during a predefined movement pattern of the subject that activates the ligament.

In at least some embodiments of the method 300, the imaging device may include two or more cameras; and the DIC produces a two-dimensional strain data vector for tracked pixels.

In at least some embodiments of the method 300, the image data is first image data, the fiber strain is a first fiber strain, and the method 300 may further include capturing, via the imaging device, second image data comprising two or more images of the ligament after the guided release; and analyzing, by the one or more processors, the second image data to determine a second fiber strain of at least a portion of the ligament. In some such embodiments, the method 300 may include, based on the second fiber strain, determining, by the one or more processors, that the guided release was successful; and providing, by the one or more processors, an indication of the successful guided release to a user interface. In some such embodiments, the method 300 may include based on the second fiber strain, determining, by the one or more processors, that the guided release was unsuccessful; and based on the second fiber strain, providing, by the one or more processors, additional guidance for performing an additional guided release of the ligament, wherein the additional guided release is performed using the perforation device to make one or more additional perforations of the ligament in accordance with the additional guidance. In some such embodiments, analyzing the second image data to determine a second fiber strain of at least a portion of the ligament may include comparing, before the guided release and after the guided release, one or more of: a length of one or more fibers of the ligament, a displacement of the one or more fibers, or a deformation of the one or more fibers.

It should be understood that not all blocks of the exemplary flow diagram of FIG. 3 are required to be performed. Additionally, the computer-implemented method 300 may include fewer, additional, and/or alternate steps than those depicted in FIG. 3.

### Additional Considerations

With the foregoing, users whose data is being collected and/or utilized may first opt-in. After a user provides affirmative consent, data may be collected from the user's device (e.g., a mobile computing device). In other embodiments, the deployment and use of ML models at a client or user device offer the benefit of alleviating concerns about privacy or anonymity by eliminating the need to transmit personal or private data to a remote server.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

The patent claims at the end of this patent application are not intended to be construed under 35 U.S. C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, any reference to “one embodiment” or “an embodiment” means that a particular element, feature, structure, or characteristic described in connection with the embodiment is included in at least one embodiment. The appearances of the phrase “in one embodiment”, “in one aspect,” and/or the like in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, the use of the “a” or “an” is employed to describe elements and components of the embodiments herein. This is done merely for convenience and to give a general sense of the description. This description, and the claims that follow, should be read to include one or at least one and the singular also includes the plural unless it is obviously evident that it is meant otherwise.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic, routines, subroutines, applications, or instructions. These may constitute either software (e.g., code embodied on a machine-readable medium) or hardware. In hardware, the routines, etc., are tangible units capable of performing specific operations and may be configured or arranged in a particular manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a specific manner or to perform certain operations described herein. Considering embodiments in which hardware modules are temporarily configured (e.g., programmed), each of the hardware modules does not need to be configured or instantiated at any one instance in time. For example, where the hardware modules comprise a general-purpose processor configured using software, the general-purpose processor may be configured as different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple such hardware modules exist contemporaneously, communication may be achieved through signal transmission (e.g., over appropriate circuits and buses) that connects the hardware modules. In embodiments where multiple hardware modules are configured or instantiated at different times, communications between such hardware modules may be achieved, for example, by storing and retrieving information in memory structures accessible to the multiple hardware modules. For example, one hardware module may perform an operation and store the output of that operation in a memory product to which it is communicatively coupled. A further hardware module may then, at a later time, access the memory product to retrieve and process the stored output. Hardware modules may also initiate communications with input or output products, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented by a processor. For example, at least some of the operations of a method may be performed by one or more processors or processor-implemented hardware modules. The performance of certain operations may be distributed among one or more processors, not only residing within a single machine, but also deployed across multiple machines. In some example embodiments, the processor or processors may be located in a single location (e.g., within a building environment, an office environment, or as a server farm), while in other embodiments, the processors may be distributed across multiple locations.

The performance of certain of the operations may be distributed among the one or more processors, not only residing within a single machine, but also deployed across a number of machines. In some example embodiments, the one or more processors or processor-implemented modules may be located in a single geographic location (e.g., within a building environment, an office environment, or a server farm). In other example embodiments, the one or more processors or processor-implemented modules may be distributed across multiple geographic locations.

Some embodiments may be described using the expressions “coupled” and “connected” along with their derivatives. For example, some embodiments may be described using the term “coupled” to indicate that two or more elements are in direct physical or electrical contact with each other. The term “coupled,” however, may also mean that two or more elements are not in direct contact with each other, but still cooperate or interact with each other. The embodiments are not limited to this context.

Upon reading this disclosure, those of skill in the art will appreciate still additional alternative structural and functional designs for the method and systems described herein through the principles disclosed herein. Thus, while particular embodiments and applications have been illustrated and described, it is to be understood that the disclosed embodiments are not limited to the precise construction and components disclosed herein. Various modifications, changes, and variations, which will be evident to those skilled in the art, may be made in the arrangement, operation and details of the method and apparatus disclosed herein without departing from the spirit and scope defined in the appended claims.

Thus, many modifications and variations may be made in the techniques, methods, and structures described and illustrated herein without departing from the spirit and scope of the present claims. Accordingly, it should be understood that the methods and apparatus described herein are illustrative only and are not limiting upon the scope of the present claims.

What is claimed:

1. A method for determining fiber strain of a ligament in a subject, the method comprising:
capturing, via an imaging device, image data comprising two or more images of the ligament;
determining, by one or more processors, a fiber strain of at least a portion of the ligament by analyzing the image data using digital image correlation (DIC); and
based on the fiber strain, providing, by the one or more processors, guidance for performing a guided release of the ligament, wherein the guided release is performed using a template having a plurality of apertures via which one or more perforations to the ligament of the subject are performed utilizing a perforation device.

2. The method of claim 1, wherein the plurality of apertures are equidistantly located within the template.

3. The method of claim 1, wherein providing the guidance comprises:
obtaining, by the one or more processors, an amount of release to achieve via the one or more perforations; and
providing, by the one or more processors, at least one of a quantity of the one or more perforations, or a location of the one or more perforations to achieve the amount of release.

4. The method of claim 3, wherein providing the at least one of the quantity of the one or more perforations, or the location of the one or more perforations comprises:
providing, by the one or more processors, an indication of apertures of the template to utilize when performing the guided release.

5. The method of claim 3, wherein obtaining the amount of release comprises:
providing, by the one or more processors, the fiber strain to a surgical robotic system; and
receiving, by the one or more processors, the amount of release from the surgical robotic system.

6. The method of claim 3, wherein obtaining the amount of release comprises:
providing, by the one or more processors, the fiber strain to a user interface presented on a display device; and
obtaining, by the one or more processors, the amount of release from the user interface.

7. The method of claim 6, wherein providing the fiber strain to the user interface comprises:
providing, by the one or more processors, a visual representation of the fiber strain of one or more fiber bundles comprising the ligament to the user interface.

8. The method of claim 7, wherein the visual representation includes color-coding indicative of local strain amounts overlaid onto the one or more fiber bundles.

9. The method of claim 1, wherein providing the guidance comprises:
analyzing, by the one or more processors, the image data to determine one or more physiological characteristics of the subject; and
based on the one or more physiological characteristics, providing, by the one or more processors, a look up table correlating an amount of release to at least one of: a quantity of the one or more perforations, or a location of the one or more perforations to achieve the amount of release.

10. The method of claim 9, wherein the physiological characteristics include one or more of the following: a length of the ligament, a thickness of the ligament, or a width of the ligament.

11. The method of claim 1, wherein the image data is captured during a predetermined movement pattern of the subject that activates the ligament.

12. The method of claim 1, wherein:
the imaging device includes two or more cameras; and
the DIC produces a two-dimensional strain data vector for tracked fiducials.

13. The method of claim 1, wherein the image data is first image data, the fiber strain is a first fiber strain, and the method further comprises:
capturing, via the imaging device, second image data comprising two or more images of the ligament after the guided release; and
analyzing, by the one or more processors, the second image data to determine a second fiber strain of at least a portion of the ligament.

14. The method of claim 13, further comprising:
based on the second fiber strain, determining, by the one or more processors, that the guided release was successful; and
providing, by the one or more processors, an indication of the successful guided release to a user interface.

15. The method of claim 13, further comprising:
based on the second fiber strain, determining, by the one or more processors, that the guided release was unsuccessful; and
based on the second fiber strain, providing, by the one or more processors, additional guidance for performing an additional guided release of the ligament, wherein the additional guided release is performed using the perforation device to make one or more additional perforations of the ligament in accordance with the additional guidance.

16. The method of claim 13, wherein analyzing the second image data to determine a second fiber strain of at least a portion of the ligament comprises:
comparing, before the guided release and after the guided release, one or more of: a length of one or more fibers of the ligament, a displacement of the one or more fibers, or a deformation of the one or more fibers.

17. A system for determining fiber strain of a ligament of a subject, the system comprising:
an imaging device configured to capture image data comprising two or more images of the ligament;
one or more processors; and
one or more non-transitory memories storing processor-executable instructions that, when executed by the one or more processors, cause the one or more processors to:
determine a fiber strain of at least a portion of the ligament by analyzing the image data using digital image correlation (DIC); and
based on the fiber strain, provide guidance for performing a guided release of the ligament, wherein the guided release is performed using a template having a plurality of apertures via which one or more perforations to the ligament of the subject are performed utilizing a perforation device.

18. The system of claim 17, wherein to provide the guidance further comprises instructions that, when executed by the one or more processors, cause the one or more processors to:
obtain an amount of release to achieve via the one or more perforations; and
provide at least one of the following: a quantity of the one or more perforations, or a location of the one or more perforations to achieve the amount of release.

19. The system of claim 17, further comprising instructions that, when executed by the one or more processors, cause the one or more processors to:
analyze the image data to determine one or more physiological characteristics of the subject; and
based on the one or more physiological characteristics, provide a look up table correlating an amount of release to at least one of the following: a quantity of the one or more perforations and a location of the one or more perforations to achieve the amount of release.

20. A non-transitory computer-readable medium having processor-executable instructions stored thereon that, when executed by one or more processors, cause the one or more processors to:
capture, via an imaging device, image data comprising two or more images of a ligament;
determine a fiber strain of at least a portion of the ligament by analyzing the image data using digital image correlation (DIC); and
based on the fiber strain, provide guidance for performing a guided release of the ligament, wherein the guided release is performed using a template having a plurality of apertures via which one or more perforations to the ligament of the subject are performed utilizing a perforation device.
